(19) United States
(12) Patent Application Publication
(10) Pub. No.: US 2008/0171849 A1
(43) Pub. Date: Jul. 17, 2008

(54) Peptide Cyclisation

(76) Inventors: Matthieu Giraud, Sion (CH); Oleg Werbitzky, Veyras (CH); Michaela Williner, Glis (CH)

Correspondence Address: NIXON & VANDERHYE, PC, 901 NORTH GLEBE ROAD, 11TH FLOOR, ARLINGTON, VA, 22203, US

(21) Appl. No.: 11/663,276

(22) PCT Filed: Sep. 20, 2005
(86) PCT No.: PCT/EP05/10133

§ 371 Date: Nov. 9, 2007

(30) Foreign Application Priority Data
Sep. 20, 2004 (EP) 04022310.9
Jul. 11, 2005 (EP) 05014954.1

(51) Int. Cl. C07K 5/12; C07K 1/00; C07F 15/00

(52) U.S. Cl. 530/317; 530/336; 530/334; 556/13

(57) ABSTRACT

A novel method for side chain cyclisation of peptides by means of lactamization is provided.

The present invention relates to a method of synthesis for a cyclic peptide, namely a cyclic peptide comprising ring closure of the carboxy group of the side chain of a one amino acid residue and the amino group of a side chain of a second amino acid residue.

On-resin cyclisation of a peptide by lactam formation in between the ω-carboxyl group of an amino acid side chain (i.e., the carboxyl group of a side chain irrespective of the carbon chain length), typically an Aspartyl or Glutamyl residue, and the ω-amino group of an amino acid side chain (i.e., the amino group of a side chain irrespective of the carbon chain length), typically a lysine residue, has been described.

Rijkers et al. (An optimized solid phase strategy—including on-resin lactamization—of Astressin, its retro-, inverso- and retro-inverso isomers, 2002, Biopolymers 63, 141-149) describe latamisation of Boc-protected 41-mer bound to Rink amide resin. Positions 30 (Glu) and 33 (Lys) are deprotected in a single step by Pd(0) catalyzed removal of allyl and alloc protection groups and are subsequently cyclised by the presence of BOP/HOBt and Hünig base in N-methyl pyrrolidone. The peptide was synthesized using a standard orthogonal protection scheme employing FMOC except for the last residue, which was Boc protected. Subsequently, the N-terminal Boc protection group was removed by acid TFA treatment, simultaneously liberating the peptide from the resin.

A disadvantage of this method is that termination of full-length peptide synthesis is a pre-requisite for subsequent cyclisation of a segment of the peptide. Hence the more valuable full-length peptide is subject to yield losses due to unwanted side-reactions (pyroglutamate formation) or incomplete allyl/alloc deprotection. Further, it is not always desirable to use an acid-labile protection group whilst on resin, since this prevents subsequent further derivatisation of the peptide on-resin, e.g. such as N-terminal blocking by acetylation. No reaction may be carried out prior to N-terminal acetylation since this renders the terminal Nα vulnerable to epimerisation at the chiral Cα.

Kates et al. (A novel, convenient three dimensional orthogonal strategy for solid-phase synthesis of cyclic peptides, 1993, Tetrahedron Letters 34:1549-1552 ) describes head-to-tail cyclisation of a decameric peptide by side chain anchoring of a C-terminal Aspartyl or Glutamyl residue to different resin handles which residue is protected at its Cα by an allyl ester protection group. After linear solid phase FMOC synthesis of the complete peptide chain, the allyl ester moiety is removed by Pd-catalysis, followed by the sequence of Nα-FMOC removal and subsequent BOP/HOBt/DIEA mediated head-to-tail cyclisation. Interestingly, the strategy where a deprotected C-terminal Aspartyl residue (Asn⁸ constituted by coupling of FMOC-Asp to a PAL handle resin) was coupled to the deprotected Nα of a N-terminal aspartyl residue, in the absence of FMOC protection group, was found to work best as compared to other synthetic strategies obtained by permutation of the starting point of synthesis along the cyclic peptide sequence.

A limiting disadvantage of this method is that it is tacitly taken into account that partial FMOC deprotection occurs as a side reaction during the main allyl deprotection step, due to the presence of nucleophilic reagents and because it does not truly affect the reaction scheme. Further completion of FMOC deprotection takes place subsequently in any event, and is required to allow subsequent head-to-tail peptide bonding. In contrast, cyclisation by means of lactamization of peptide side chain functionalities only is crucially dependent on preserving complete protection of the Nα.

Blankemeyer et al. (1988, Tetrahedron Lett. 29, 5871-5874) described the synthesis of several protected peptide fragments on cellulose discs containing 4-(4′-methoxytrityloxy)-but-2-enyloxy-hexanoic acid as an allylic handle to the solid phase, further employing FMOC chemistry. Cleavage of the allyl ester linker was achieved by treatment with Pd(PPh₃)₄ in dry THF followed by the addition of a solution of HOBt in THF.

Another method of peptide side chain cyclisation is devised according to the present invention, avoiding the disadvantages of the prior art and being particularly useful for cyclisation of peptides comprising aspartyl side chains.

According to the present invention, a cyclisation method for a peptide comprises the steps of
a. deprotecting the peptide from allyl-type protecting groups, which peptide is protected with a base-labile protection group at its Nα and which peptide further comprises at least one allyloxy-carbonyl-protected amino-function of a lysine side chain (i.e. the ε-amino group) or of an analogue of such lysine side chain and further comprises at least one allyl-ester-protected ω-carboxyl group of a glutamyl (i.e. a β-carboxy group) or aspartyl side chain (i.e. a γ-carboxy group) or of an analogue of such side chains, whilst retaining the base-labile protection group on the Nα,
b. cyclising the peptide by lactamisation of said deprotected side chains in the presence of a weak base reagent, and
c. deprotecting the peptide from the base-labile protection group at its Nα,

with the further proviso, that said allylic protection groups may be unsubstituted or may be further substituted with alkyl or aralkyl that in itself may be unsubstituted or further substituted with halogen or alkoxy. In a preferred routine embodiment, the standard unsubstituted Alloc (i.e. allyloxy-carbonyl or prop-2-enyl-oxy-carbonyl) protection group is employed for protection of the ε-amino-function and the γ-carboxy group is protected by esterification with allyloxy (propen-2-oxy).

In the present context, the ‘ω-carboxyl group’ of an amino acid side chain is understood as being the ‘termina’l carboxyl group of a side chain irrespective of the carbon chain length, and the ‘ω-amino group’ of an amino acid side chain is understood as being the ‘terminal’ amino group of a side chain irrespective of the carbon chain length. Analogues thereof may well be e.g. side chain isomers thereof. For example, a γ- or δ-amino isomer of lysine is a suitable analogue of natural lysine. In the context of the present invention, the term ‘side chain’ in respect to an amino acid or amino acid derivative is used in compliance with the respective IUPAC-IUB definition (International Union of Pure and Applied Chemistry and International Union of Biochemistry/Joint Commission on Biochemical Nomenclature, “Nomenclature and Symbolism for Amino Acids and Peptides”, Pure Appl. Chem, 56, 595-624 (1984)).

This reaction path has not been described before. Unexpectedly, it has been found that integrity of the FMOC group is largely preserved after allyl-deprotection, in particular when allyl-deprotection is carried out on solid-phase bound peptide, and in consequence cyclisation is also carried out on the solid-phase bound material. Preferably, after completion of the reaction sequence, peptide chain elongation is continued, more preferably is continued in a solid-phase mode, since having the advantage that the preceding cyclisation step would then not require application of limiting dilution conditions for favoring intramolecular cyclisation over intermolecular reactions. The present reaction will hence be particularly beneficial where multiple glutamyl and/or aspartyl and lysine residues, or their analogues such as e.g. nor- or homo-lysine, are present in the final peptide chain whilst only a specific pairing of those residues is scheduled for cyclisation on a firstly synthesized portion of the final full-size peptide chain. This regional-lactamisation approach will be particularly beneficial when the full-size peptide comprises several subsequent subdomains or peptide loop structures for bioactive peptides that are to be stabilized by side-chain cyclisation. Lactamization is a more stable, less redox-vulnerable option than naturally stabilized disulfide-bridging or non-natural, chemical analogues thereof employing amino-acid analogues. The latter functionalities usually prove to be more immunogenic in vivo, whereas the present invention allows use of natural amino acid analogues as a superior option.

In a preferred embodiment, the peptide contains just one allyl-protected lysine residue and just one allyl-protected aspartyl or glutamyl residue, and hence there is one and only one bonding option upon cyclisation, giving rise to a homogenous product.

In contrast to prior art methods, the peptide according to the method of the present invention can be utilized for further N-terminal peptide elongation, optionally and preferably while on solid phase, be it in the sequential, stepwise mode of adding or modifying amino acid residues or other groups, such as by addition of new N-protected amino acids or be it by N-terminal condensation reaction with another peptide.

In a further particularly preferred embodiment, the N-terminal residue of the protected peptide having a base-labile protection group at its Nα is said at least one allylester-protected aspartyl or glutamyl residue or analogue thereof. A seldom appreciated problem with such N-terminally protected, preferably FMOC protected, and simultaneously side chain-protected amino acids is that they are prone to a base-catalyzed side reaction upon FMOC deprotection, giving rise to either aspartimide or glutarimide. It is an undesired shortcoming of present standard solid phase chain elongation synthesis employing FMOC protection groups that is usually believed to be avoided by protection of γ-carboxylic acid groups. However, this is not true as has been shown by the reports of Kates et al. and others (Kates et al., Lett. Pept. Sci., 1995, 1, 213; Nicolas et al., Tetrahedron Lett. 1989, 30,497) for different protection groups for γ-carboxy functionalities. The same holds true according to the present inventor's observation for allyl ester protection of the γ-carboxy functionality of an FMOC protected, N-terminal aspartyl residue under standard solid phase conditions for FMOC deprotection. Its precise quantitative extent is highly sensitive to slight changes in processing time and conditions, giving rise to undesired variation of product yield:

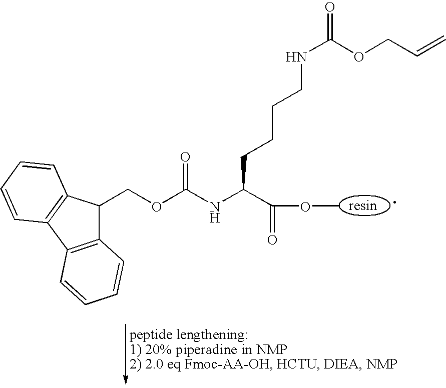

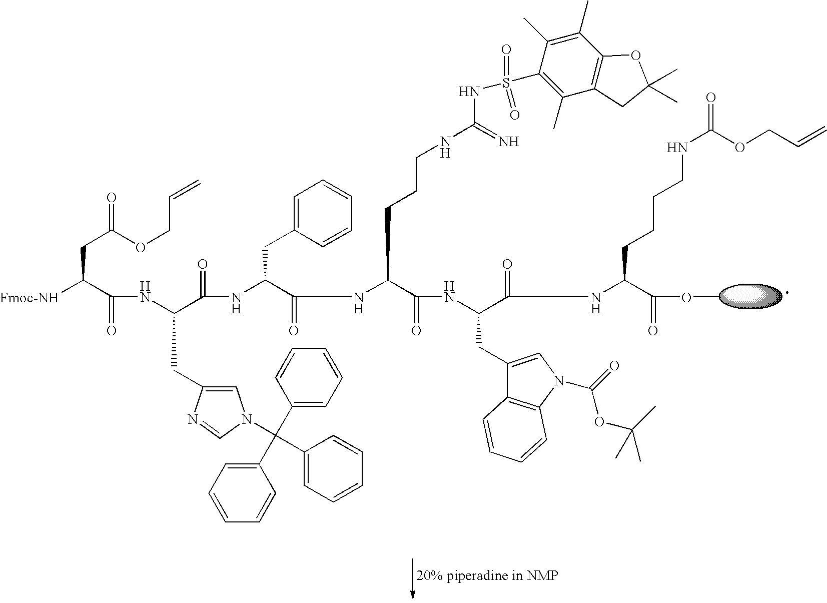

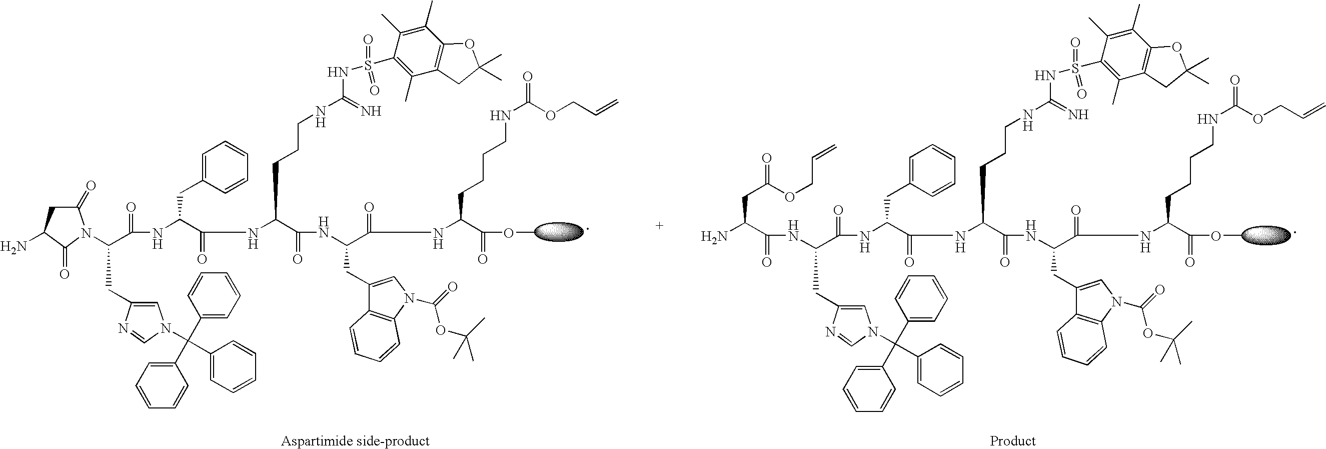

Accordingly, with the method of the present invention, it is now possible to avoid this unproductive side reaction by immediate side chain cyclisation of such N-terminal, FMOC and allylester-protected aspartyl or glutamyl residue. Again, FMOC protection is preserved, effectively preventing aspartimide formation. Thus subsequent FMOC deprotection followed by further chain elongation is possible without glutarimide or aspartimide formation, due to the prior lactamization.

More preferably, the N-terminal allylester-protected acidic residue is an aspartyl residue. Aspartimide formation has a higher reaction rate than glutarimide formation and is usually the more dominant side reaction. It is particularly favored in the dipeptide sequence L-Asp-L-X where X is Gly, Ser, Thr or Asn, and as we report here for the first time, where X is His, easily yielding up to 30 or 40% aspartimide. In particular the -Asp-Gly- dipeptide is highly prone to aspartimide formation; usually, this requires use of an additional HMB protection group on the glycine, providing steric hindrance of aspartimide formation as described in Packman et al., 1995, Tetrahedron Lett. 36, 7523. However use of Gly(HMB) dipeptides is extremely expensive and hence is sought to be avoided. Thus in these instances the present invention offers even a further advantage over the prior art.

The allyl and/or alloc deprotection step (in the following, allyl deprotection for short) may be carried out by the methods known in the art, such as e.g. hydrostannolysis with Bu₃SnH or treatment with tetrakis-triphenylphosphine palladium (0) [Pd(PPh₃)₄] in THF at essentially neutral conditions in the presence of a nucleophile such as e.g. morpholine, dimedone, N-methylaniline, HOBt, borhydride or N,N′-dimethylbarbituric acid as an allyl acceptor. Variants of said methods employing different pH exist, but of course care must be paid in view of the pH sensitivity of resin linkage or handle groups and the base sensitive protection group.

The allyl deprotection preferably employs catalysis with allyl-group reactive palladium complexes, preferably with Pd(0) complexes, preferably with palladium complexes having C₁-C₁₀ trialkylphosphite, C₃-C₁₀ tricycloalkylphophite or triarylposphine or triheteroarylphosphine ligands, wherein said aryl or heteroaryl may be further substituted with electron-donating substituents or is unsubstituted, more preferably with palladium complexes having phenylphosphine ligands wherein the phenyl may be further substituted with C₁-C₅ alkyl, preferably wherein the phenyl is tolyl or xyloyl, more preferably is phenyl, 2,4-xyloyl or o-tolyl. Preferably, said phosphine ligands are mono-phosphine ligands, more preferably non-chelating, monovalent ligands. Although the palladium complexes preferably are mono-palladium complexes, the term complex is to be understood as to also comprise di-palladium or higher palladium complexes, though mono-palladium complexes are preferred. Using tetrakis (triphenylphosphine)-palladium [Pd(PPh₃)₄], in the presence of an allyl acceptor or scavenger, or using corresponding Pd (P[ortho-tolyl]₃)₂ or Pd (P[2,4-xyloyl]₃)₂ complexes or mixed complexes possibly having both tri-phenylphosphine and tri-(o-tolyl)phosphine ligands is mostly preferred in the present invention. As compared to triphenylphosphine, methylphenylphosphine ligands and especially the tri-o-tolylphosphine ligands improve the catalytic reaction rate, further allowing lowering the total amount of precious metal catalyst used whilst maintaining optimal yields. For reference to Pd(0)-catalyzed allyl and allyloxy deprotection, compare Jeffrey et al., J. Org. Chem. 1982, 47:587-590. As has been described in Jeffrey, it is also feasible and perfectly customary in the art to constitute the complexes of the present invention as an exchange catalyst in situ, namely by mixing less stably coordinated Pd-complexes with the preferred ligands of the present invention. Hence examples of suitable catalyst complexes apart from the strongly preferred Pd(PPh₃)₄ are: PdCl₂(PPh₃)₂/PPh₃, PdCl₂(PPh₃)₂/P(oTol)₃, Pd(DBA)₂/P(oTol)₃ or Pd[P(oTol)₃]₂ (Organometallics 1995, 14(6):3030-3039), Pd(OAc)₂/triethyl-phosphite, Pd(OAc)₂/PPh₃ or Pd(OAc)₂/P(oTol)₃. The presence of less coordinating anionic, basic ligands such as acetate, benzoyl or amines (e.g. Huenig base) in the initially added Pd(0) complexes allows constituting the preferred complexes in situ due to the presence of suitable free ligands such PPh₃ or P(oTol)₃ [oTol=ortho-tolyl-]. It is not mandatory in general to add free ligand for exchange with the catalyst though, especially when using highly active arylic phosphine palladium complexes from the start—this option is noteworthy in case of zerovalent Pd(PPh₃)₄ which was originally used by Jeffrey et al. (supra) with extra PPh₃ added to the reaction broth. The general mechanism of Pd-catalyzed deprotection in the presence of an allyl acceptor is an acyl group transfer reaction (transacylation), as is well known in the art. Hence the choice of allyl acceptor reagent or scavenger is likewise important for achieving quantitative deprotection under mild reaction conditions whilst avoiding unwanted side reactions. A suitable scavenger is any nucleophil such as e.g. morpholine, dimedone, N,N-dimethylbarbituric acid, methylaniline or thiosalicylic acid.

Suitable catalytic amounts of Pd(0) complexes preferably are used in an amount of 0.005 eq. to 0.5 eq. catalyst as compared to educt, more preferably are used in an amount of 0.01 up to 0.1 eq. of catalyst, most preferably are used in an amount of 0.015 eq. up to 0.07 eq. of catalyst. Preferably, reaction temperature is in between 10-60° C., more preferably in between 30-50° C., most preferably at about 40° C.

In one preferred embodiment of the present invention, amine-borane complexes are employed in at least 1.5 to 2-fold excess per allyl function of the educt as the nucleophilic allyl group scavenger as described in Gomez-Martin et al., J. Chem. Soc., Perkin Trans. 1(1999): 2871-2874, Nα-Alloc temporary protection in solid-phase peptide synthesis—use of amine-borane complexes as allyl group scavengers. Depending on the exact composition of the amine moiety in the complex, high conversion rates along with very short reaction time can be realized, for instance with t-Bu-NH₂.BH₃, Me₂NH.BH₃ or NH₃.BH₃. Quaternary amines are excluded from the present definition of suitable complexes, whereas the amine may be preferably a primary or secondary alkyl amine or may be ammonia. In an optional, even more preferred embodiment, said Pd(0) catalyzed allyl-deprotection is carried out as a hydrosilylolysis in the presence of the hydride donor phenyl-trihydrosilane PhSiH₃ or functional derivatives thereof in an aprotic, polar organic solvent such as e.g. dichloro-methane, as has been essentially described by Dessolin et al., Tetrahedron Lett. 1995, 36: 5741-5744, New allyl group acceptors for palladium catalyzed transacylation of allyl carbamates. Preferably, a phenyl-hydrosilane reagent of the generic formula R1—PhₙSiHₘ is employed as the allyl acceptor that is usually in excess of at least 1.5 to 2 eq. wherein R1 is a substituent at the aromatic core and is aryl, alkyl or aralkyl, n is 1 or 2 and m is 2 or 3, most preferably where the allyl acceptor is PhSiH₃.

Both phenylsilanes and suitable amine-borane complexes allow of rapid, complete deprotection in the range of <1 h, typically at about 20-40 min. Accordingly, they allow mild and short reaction conditions. Other allyl scavengers may require considerable longer reaction times.

Though technically perfectly feasible reagents, it must be mentioned that both classes of above allyl-scavengers are noxious reagents that may raise concerns from an environmental and working safety point of view. In another strongly preferred embodiment that is particularly attractive for industrial scale manufacture, an organic sulphinate is used as an allyl group acceptor reagent (Honda et al., Deprotection of allyl groups with sulfinic acids and palladium catalyst, J. Org. Chem. 1997, 62, 8932-8936). Surprisingly, in the reaction of the present inventions, this reaction allows particular high product yields, possibly due to the stability of the adducts thus formed and the mild reaction conditions. Examples of such are 4-chloro-3-nitrobenzene-sulfinate, 2-thiophenesulfinate, benzene-sulfinate, p-tolylsulfinate or hydroxymethansulfinate (also known in form of its sodium or zinc salt as Rongalit™, the traditional trivial name for such salts having been formaldehyde sulfoxylates). The organic sulfinate Rᵢ—SO₂⁻ (or Ri-S(O)O⁻, respectively) may comprise any type of further substituted organic residue, alkyl, cylcoalkyl, aryl, heteroaryl, or aralkyl (cf. Honda et al., supra). It may be added in its salt or acid forms. Even the presence of competing nucleophilic moieties in said sulfinate is possible, though less preferred. Preferably, for optimal yields attainable, the radical Ri is an optionally further substituted phenyl radical, more preferably is a single or multiple, alkyl-, alkyloxyalkyl- or alkyloxy-substituted phenyl radical, and most preferably is phenyl, xyloyl, or tolyl, especially p-tolyl. Use of sulfinates also allows use of tri-alkyl or cycloalkyl phosphites ligand complexes with good yields, in addition to the more preferred triarylic phosphine complexes.

The lactamisation reaction is carried out in an essentially similar manner to standard peptide chain elongation reactions in the presence of base-labile amine-protection groups such as FMOC, except that the base labile group is carried on the same peptide rather than adding a further N-terminally protected amino acid as in chain elongation. Of course, the same chemistry may subsequently be employed for further chain elongation in an optional step d. after cyclisation and deprotection. Both lactamization and chain elongation require a coupling reagent and eventually a coupling additive, depending on the type of primary coupling reagent or auxiliary.

Coupling reagents for peptide synthesis are well-known in the art (see Bodansky, M., Principles of Peptide Synthesis, 2^{nd} ed. Springer Verlag Berlin/Heidelberg, 1993; also see discussion of role of coupling additives or auxilliaries therein). Coupling reagents may be mixed anhydrides (e.g. T3P: propane phosphonic acid anhydride) or other acylating agents such as activated esters or acid halogenides (e.g. ICBF, isobutyl-chloroformate), or they may be carbodiimides (e.g. 1-ethyl-3-(3-dimethylaminopropyl)-carbodiimide), activated benzotriazine-derivatives (DEPBT: 3-(diethoxyphosphoryloxy)-1,2,3-benzotriazine-4(3H)-one) or uronium or phosphonium salt derivatives of benzotriazol.

In view of the best yield, short reaction time and protection against racemization during chain elongation, more preferred is that the coupling reagent is selected from the group consisting of uronium salts and phosphonium salts of benzotriazol capable of activating a free carboxylic acid function where the reaction is carried out in the presence of a base. Suitable and likewise preferred examples of such uronium or phosphonium coupling salts are e.g. HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), HAPyU (O-(benzotriazol-1-yl)oxybis-(pyrrolidino)-uronium hexafluorophosphate.

Preferably, the base reagent is a weak base whose conjugated acid has a pKa value of from pKa 7.5 to 15, more preferably of from pKa 7.5 to 10, with the exclusion of an α-amino function of a peptide or amino acid or amino acid derivative, and which base preferably is a tertiary, sterically hindered amine. Examples of such and further preferred are Hünig-base (N,N-diisopropylethylamine), N,N′-dialkylaniline, 2,4,6-triallcylpyridine, 2,6-trialkylpyridine or N-alkyl-morpholine with the alkyl being straight or branched C₁-C₄ alkyl, more preferably it is N-methylmorpholine or collidine (2,4,6-trimethylpyridine), most preferably it is collidine.

The use of coupling additives, in particular of coupling additives of the benzotriazol type, is also known (see Bodansky, supra). Their use is particularly preferred when employing highly activating uronium or phosphonium salt coupling reagents. Hence it is further preferred that the coupling reagent additive is a nucleophilic hydroxy compound capable of forming activated esters, more preferably having an acidic, nucleophilic N-hydroxy function wherein N is imide or is N-acyl or N-aryl substituted triazeno, most preferably the coupling additive is a N-hydroxy-benzotriazol derivative (or 1-hydroxy-benzotriazol derivative) or is an N-hydroxy-benzotriazine derivative. Such coupling additive N-hydroxy compounds have been described in WO 94/07910 and EP-410 182 and the respective disclosure is incorporated by reference hereto. Examples are e.g. N-hydroxy-succinimide, N-hydroxy-3,4-dihydro-4-oxo-1,2,3-benzotriazine (HOOBt), 1-hydroxy-7-azabenzotriazole (HOAt) and N-hydroxy-benzotriazole (HOBt). N-hydroxy-benzotriazine derivatives are particularly preferred, in a most preferred embodiment, the coupling reagent additive is hydroxy-3,4-dihydro-4-oxo-1,2,3-benzotriazine. Ammonium salt compounds of coupling additives are known and their use in coupling chemistry has been described, for instance in U.S. Pat. No. 4,806,641.

In a further particularly preferred embodiment, the uronium or phosphonium salt coupling reagent is an uronium salt reagent and preferably is HCTU, TCTU or HBTU and even more preferably is used in the reaction in combination with N-hydroxy-3,4-dihydro-4-oxo-1,2,3-benzotriazine or a salt thereof. This embodiment is mainly preferred for use in chain elongation step of peptide synthesis after removal of the base-labile Nα-protection group, but may as well be used for lactamization reaction during side-chain cyclisation.

In the context of the present invention, it is to be noted that HCTU and TCTU are defined as to be encompassed by the term ‘uronium salt reagent’ despite that these compounds and possible analogues have been shown to comprise an isonitroso moiety rather than an uronium moiety by means of crystal structure analysis (O. Marder, Y. Shvo, and F. Albericio “HCTU and TCTU: New Coupling Reagents: Development and Industrial Applications”, Poster, Presentation Gordon Conference February 2002), an N-amidino substituent on the heterocyclic core giving rise to a guanidium structure instead. In the present context, such class of compounds is termed the ‘guanidium-type subclass’ of uronium salt reagents according to the present invention.

In a further particularly preferred embodiment, which is mainly used for the lactamization reaction, the coupling reagent is a phosphonium salt of the benzotriazol such as e.g. BOP, PyBOP or PyAOP.

Deprotection of the base labile Nα may be carried out as routinely done in the art, e.g. with 20% piperidine in N-methyl morpholine.

A further object of the present invention is a cyclic peptide of formula II or III having an Nα that is protected with a base-labile protection group,

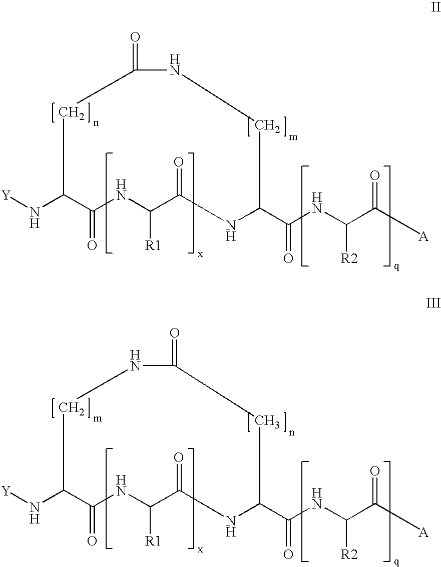

wherein Y is the base-labile protection group, n=1-10, preferably n=1 or 2, most preferably n=1, further wherein m=1-15, preferably m=3 to 6, most preferably m=3, further wherein x=1-200 and q=0-200 wherein R1 and R2 each are, independently, a natural amino side chain or non-natural derivative thereof, which side chain further may comprise a protection group with the exception of allylether and allyloxycarbonyl protection groups, and wherein A is a resin or resin handle or wherein optionally R2 may also be a natural amino side chain or non-natural derivative thereof which side chain is bonded to a resin or resin handle via an ether, thioether, ester, thioester, amido or secondary or tertiary amino moiety with the proviso that then A is selected from the group consisting of OH, NH₂, NR′1H or NR′1R′2, OR′3 with R′1 and R′2 being independently C₁-C₄ alkyl and R′3 being a protection group other than or with the exception of allyl groups, preferably with R′3 being tert.butyl or pentafluorophenyl.

A side chain group, such as for example R1₍ₓ₌₁₎, is not to be construed so as to refer to a single type of optionally protected amino acid side chain; that is, each residue R1(1), R1(2), and so on may be unique or may be the same as at least one other residue. The same applies of course to radicals R2_{(q=1)}, R2_{(q=2)}, and so on.

The resin or resin handle composite entity may in principle be any resin employed for synthesis, such as for example a polystyrene-divinylbenzene resin as used by Merrifield along with hydroxybenzyl-phenyl integral linker moieties or by Wang with hydroxy-benzyl-p-benzyloxy moieties, such as for example moieties to which e.g. more acid-labile linkers may be further grafted, or alternatively the latter linkers may be integrally or directly linked to the resin. In principle, a solid phase resin for use in synthesis necessarily comprises at least an integral linker or handle which is part of the solid phase core material; such linker or handle may be considered as an immobilized protection group (Guillier et al., Chem. Rev. 100,2091-2157, 2000). Examples are e.g. Sieber resin, related xanthenyl type PAL handle resins, Rink amide resin, Rink acid resin, more complex PEG-grafted polystyrene resins such as tentagel-based Novasyn TG (Novabiochem, Merck Biosciences, Germany) which are available with different grafted handles such as 2′-chloro-trityl, or resins that are constituted by grafting functional handles onto matrix material such as silica gels. Preferably, where the resin is a trityl resin or resin handle, such resin is a 4-methoxy or 4,4′-dimethoxy-trityl resin. Resins as used in the present invention are of standard mesh size, which is about 50-500 mesh, more preferably 100 to 400 mesh. A resin or solid-phase R′″ as shown in formula IV is to be construed as to comprise a crosslinked, polymeric matrix material which may be bound to the handle moiety specified in formulas IV to VII by way of any kind of chemically inert alkyl, alkyloxy, aryloxy or alkylester spacer or linker which is to be considered an integral part of R′″. However, it should be noted that apart from impacting the conditions of cleavage from the resin, the chemical nature of the resin material and in particular the chemical nature of the handle group may well influence synthetic efficiency of coupling and especially lactamisation reactions in a yet poorly understood fashion. The yields of mature peptide at the on-resin stage may differ depending on the type of resin or resin handle employed. For this reason, in an preferred embodiment according to the present invention the resin or resin handle is of formula IV as set forth in the claims in detail, more preferably of formula VI and most preferably of formula VII as set forth in the claims in detail. Examples of such resins or resin handles are (4-methoxyphenyl)-methyl- and (4-methylphenyl)-methyl-polystyrene (Atkinson et al., 2000, J. Org. Chem. 65, 5048), resins in O- or N-linkage to the peptide moiety and their PEG-resin derivatives, respectively. Further examples are e.g. acid-labile HMPB-MBHA o HMPB-BHA resin (Sieber et al., 1987, Tetrahedron Lett. 28, 6147), acid-labile Rink amide resin or Rink acid resin (Rink et al., 1987, Tetrahedron Lett. 28,3787). The term ‘acid-labile’ refers to essentially quantitative cleavage in 2-10% TFA in dichloromethane at ambient temperature for at least an hour. Surprisingly, using such preferred resins having the diphenyl-methyl structural core motif allow for more efficient coupling reaction during linear synthesis and lactamisation; notably, such resins also allow a lower reaction temperature of 15-25° C. as compared to the standard 40° C. required for efficient coupling on e.g. tritylresins.

Preferably radical A (as given e.g. in formula II or III) comprises a resin handle or resin linkage moiety with the exception of resin handles comprising an allyl-oxycarbonyl moiety. More preferably, such resin or resin handle is of formula IV

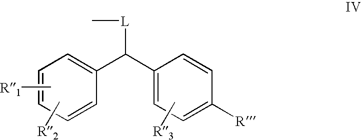

wherein R′″ is a resin, and R″1, R″2, R″3 are, independently, hydrogen, C₁-C₄ alkyl or C₁-C₄ alkoxy, and may be the same or different with the provisio that only one of R″1, R″2 may be hydrogen, and wherein L is oxygen, sulfur, nitrogen or is of formula V

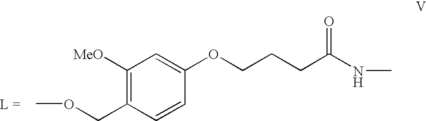

Again even more preferred is that resin or resin handle is of formula VI, the above definitions for radicals R′″, R″1 and R″2 applying with the proviso that L is selected from the group consisting of oxygen and nitrogen,

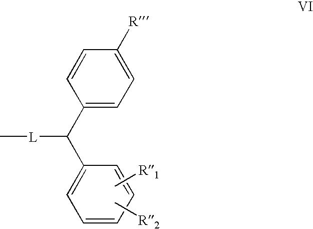

Again even more preferred is that the resin or resin handle is of formula VII, the above definitions for radicals R′″, R″1 and R″2 applying with the proviso that L is selected from the group consisting of oxygen and nitrogen,

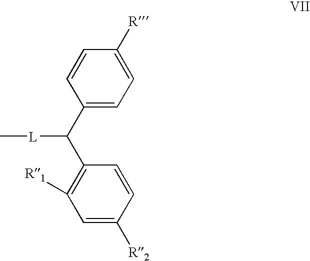

In a further even more preferred embodiment, it is preferred that R″1, R″2 are, where X is oxygen, independently hydrogen, methyl or methoxy with the provisio that only one of R″1, R″2 may be hydrogen, and where X is nitrogen, independently are methyl or methoxy, preferably are methoxy. Even more preferably then, X is oxygen, R″1 is hydrogen and R″2 is methyl or methoxy and preferably A is a resin or resin handle. Most preferably, R″2 is methyl.

Most preferably, the peptide according to the present invention is carboxy terminally coupled to the resin or resin handle (A=resin or resin handle in formula IV).

Preferably, the peptide's sequence according to the present invention is Ac-Nle-cyclo(Asp-His-D-Phe-Arg-Trp-Lys) or Nle-cyclo(Asp-His-D-Phe-Arg-Trp-Lys), a lactam bond being in place between the Asp and Lys side chains as shown in table 1. Said peptide is a pharmaceutically active melanocortin receptor-specific peptide useful for treatment of sexual dysfunction, including male erectile dysfunction and female sexual dysfunction in humans.

In another preferred embodiment, the peptide's sequence according to the present invention consists of or comprises at least the partial sequence cyclo(Asp-His-Phe-Arg-Trp-Lys), wherein the Phe residue may also be substituted by D-Phe, or the respective D- or L-isomer of pF-Phe, Phe(4-Br), Phe(4-CF₃), Phe(4-Cl), Phe(2,4-diCl), Phe(3,4-diCl), Phe(3,4-diF), Phe(4-I), Phe(3,4-di-OMe), Phe(4-Me) or Phe(4-NO₂). These modifications with non-natural derivatives of Phe modulate pharmaceutical activity of said peptide. Likewise, in the above sequence, the Arg may also be substituted with D-Arg, or the respective D- or L-isomer of Arg(NO₂), Arg(Tos), Arg(Pbf), Arg(Mtr), Arg(Me) or Arg(Pmc).

The arginine side chain may be preferably covalently protected during synthesis e.g. with tosyl, benzyloxycarbonyl, pentamethylenchromanesulfonyl (Pmc), pentamethyldihydrobenzofuransulfonyl (Pbf), 4-methoxy-2,3,6-trimethylbenzenesulfonyl (Mtr) and its 4-tbu-2,3,5,6-tetramethyl homologue (tart), adamantyloxycarbonyl or Boc. Pmc, Pbf, Mtr or Tart are strongly preferred for protecting Arg, most preferably it is Pbf.

Trp is preferably protected during synthesis with Boc. Optionally, it may be N-protected with formyl, sym-mesitylene-sulfonyl.

His is preferably protected by N-trityl protection group. Optionally, though less preferred, it may be likewise N-protected with Boc, methyltrityl or tosyl.

### EXPERIMENTS

A fully protected FMOC-version of the peptide Ac-Nle-cyclo(Asp-His-D-Phe-Arg-Trp-Lys)—whose properties were described in WO 01/000224 drawn prominently to said peptide only—was newly synthesized on different resins, e.g. on 2-chlorotrityl-polystyrene resin, and processed according to the present invention as shown in table 1. N-terminally, in the first step of the reaction scheme shown in table 1, the sequence is FMOC-Asp(OAll)-His(Trt)- . . . The Lys(Alloc) and the Asp(OAll) are five residues spaced apart, with bulky side chains such as e.g. Arg(Pbf) protruding in between. In the table below, Nle is Norleucine.

TABLE 1
model peptide and reaction pathway

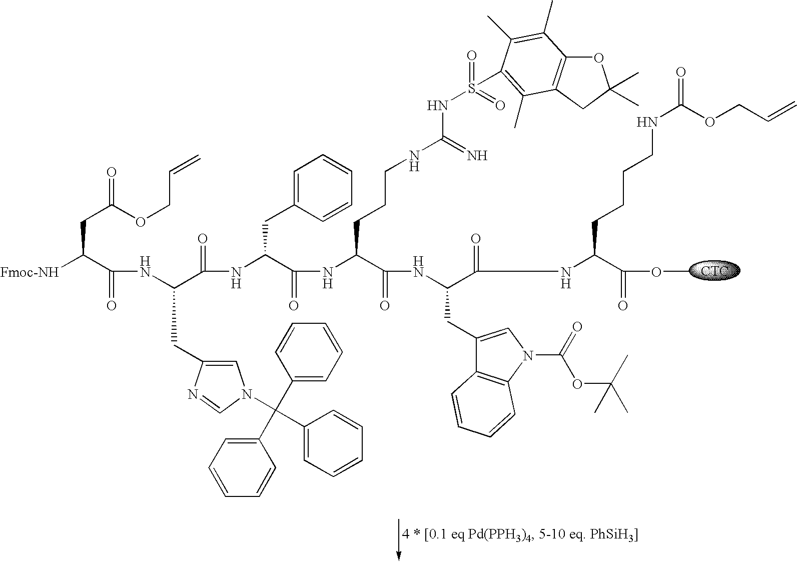

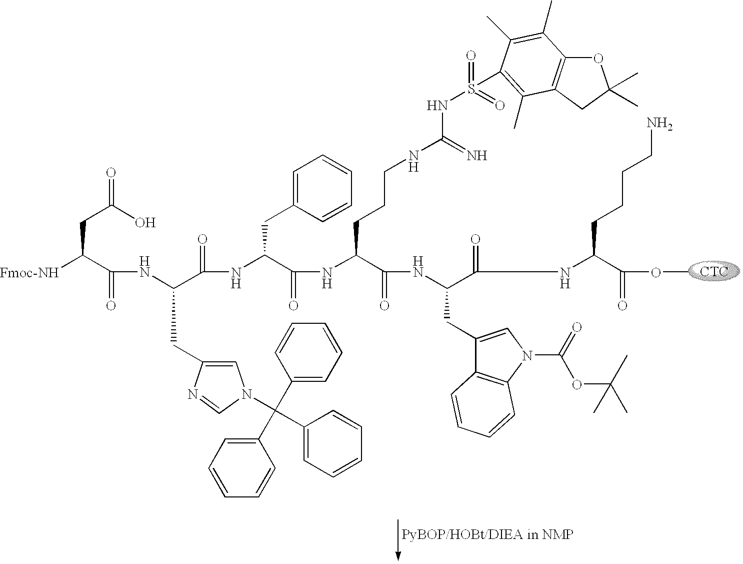

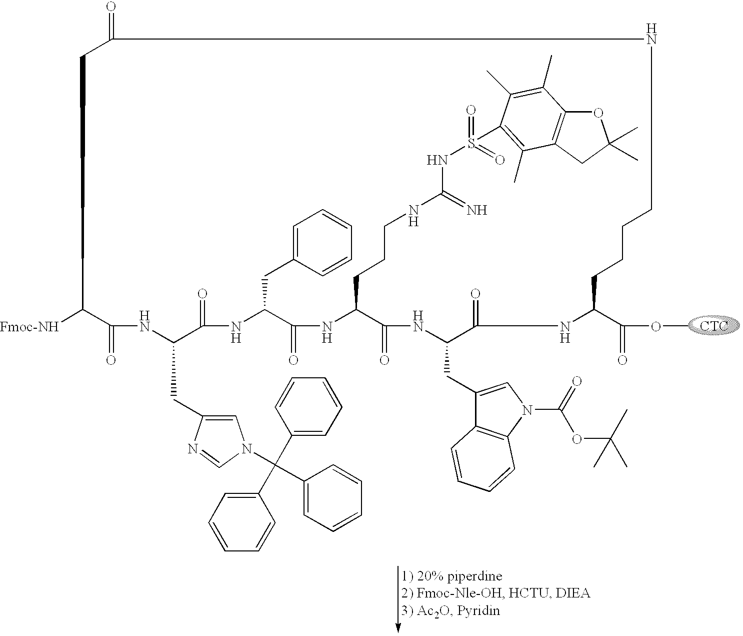

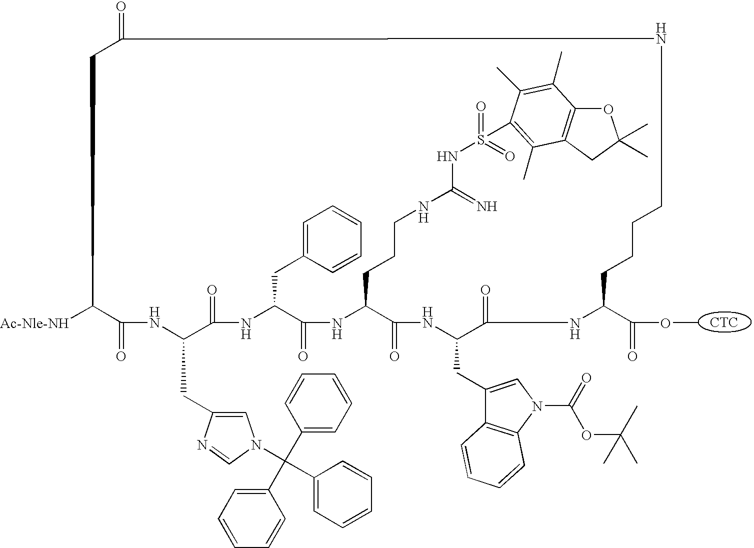

1.1 Allyl/Alloc Deprotection of 2-CTC Resin Conjugated Model Peptide

0.1 eq. of Pd(PPh₃)₄ was solubilized in DCM in the presence of PhSiH3 or diaminoborane (5 eq.).

This solution was added to the CTC-resin carrying 1 eq. (35 g resin, loaded at 0.44 mmol/g) of the allyl/alloc protected starting peptide of table 1. After max. 15 min of reaction at room temperature under steady nitrogen bubbling, the mixture was filtered, and the recovered resin subjected three more times to the same treatment, always with the same amount of catalyst and 5 eq. of phenylsilane as scavenger per cycle.

Washing step: Afterwards the resin was consecutively washed with
N-methyl-pyrrolidone (NMP) (3 times)
Dichloro-methane (DCM) (3 times)
0.5% DIEA (Hünig base) in DCM (3 times)
Sodium diethyldithiocarbamate trihydrate 0.02 N in NMP
NMP (5 times)

1.2 Cyclisation: Lactamisation of Side Chains

1 eq. of PyBOP and 1.8 Eq. of HOBt were solubilized in NMP and added to the Asp/Lys-unprotected FMOC peptide in the presence of 4 Eq. of DIEA. The reaction was stirred for 2 hours at room temperature. The lactamized peptide was recovered by filtration and washed with NMP.

HPLC reverse phase analysis of the (C-18 column, loading of about 0.1 g/ml solution in 0.1% TFA 70% water/30% acetontrile, elution with 0.1% TFA acetonitrile gradient) intermediate peptide samples released with 2% TFA from the CTC resin showed that conversion was almost complete and that >90% of the lactamized, protected peptide retained the FMOC moiety.

1.3 FMOC Deprotection and Peptide Elongation

FMOC deprotection was carried out with 20% piperidine in NMP. Subsequently, chain elongation was carried out for 30 min. in DMF at about room temperature (40° C.) for 1 h with FMOC-L-Nle (1 eq.) in the presence of 1 eq. HCTU and 3 eq. HOOBt, 3 eq. DIEA.

After filtering off the resin-bound product and washing with DMF, the FMOC moiety on Nle was removed with 20% piperidine in NMP, and an N-terminal acetyl group incorporated by incubation in pyridine with about 1.5 eq. of acetanhydride for 1-2 h at room temperature.

After filtering off the resin, the peptide was released and globally deprotected by concentrated TFA treatment. The total yield of acetylated, lactamized peptide based on crudely purified product was roughly determined to amount to ˜60%.

The need for further, final on-resin derivatization with an acylating reagent further demonstrates the utility of the present invention, namely to carry out lactamization in the presence of but without compromising a base-labile Nα-protection group. Using acid-cleavable N-protection groups such as Boc would have created further problems here at least for on-resin processing.

2.1 Allyl/Alloc Deprotection of Bromo-(4-Methylphenyl)Methyl Polystyrene Resin Conjugated Model Peptide in the Presence of an Aminoborane

Essentially experiment 1.1 was repeated, using the (CH₃)₂NH.BH₃ as scavenger, except for exchanging the CTC resin for Bromo-(4-methylphenyl)methyl polystyrene (approx. 200 mesh, 1.2-2.2 mmol/g) (CBL Patras, Greece). Catalyst was solubilized in AcOH/DMF (approx. 4:1), whilst deprotection reaction was carried out in DMF (5 times resin volume) at 40° C. for 30 min. Steps 2.2 and 2.3 were carried out as 1.2, 1.3 above, except that cleavage was conducted in 5% TFA in dichloromethane in the presence of 2% TIS. Yield of acetylated, mature peptide amounted to 72.8% (analytical grade purity).

3.1 Allyl/Alloc Deprotection of Bromo-(4-Methylphenyl)Methyl Polystyrene Resin Conjugated Model Peptide with Exchange Catalyst Pd(Oac)₂/P(oTol)₃

Essentially reaction 2.1 was repeated, but with the following modifications: Instead of 0.1 eq. of Pd(PPh₃)₄, 0.05 eq. of Pd(Oac)₂ in the presence of 0.05 eq. of ortho-tolylphosphine were used. Further, 2.2 eq. of sodium p-tolylsulfinate were added as scavenger. Even after 2 hours, conversion had only taken place in trace amounts; raising the temperature to 60° C. did not change that.

4.1 Allyl/Alloc Deprotection of Bromo-(4-Methylphenyl)Methyl Polystyrene Resin Conjugated Model Peptide with Exchange Catalyst Pd(P[oTol]₃)₂ and with Sulfinate

Catalyst Pd(P[oTol]₃)₂ was obtained as described in Paul et al., Organometallics (1995), 14(6), 3030-3039, Paul et al. further describing obtaining related Pd(P[2,4-Xyloyl]₃)₂. Catalyst was solubilized in AcOH/DMF(2:1). The reaction was carried out essentially as described in 2.1, but with using 0.05 eq. of Pd(P[oTol]₃)₂. 2.2 eq. of sodium p-tolylsulfinate were added as scavenger under steady nitrogen bubbling. Reaction took place for 30 min. at 40° C. The reaction proceeded smoothly, with the analytical yield of cleaved, acetylated peptide amounting to 82% after step 4.2, step 4.3 having been carried out as in section 2 above. Extending the reaction time of allyl/alloc deprotection to 150 min. did not significantly increase yield (yield: 84.5%). Lowering the amount of catalyst by about half slowed the reaction kinetic considerably.

5.1 Allyl/Alloc Deprotection of Bromo-(4-Methylphenyl)Methyl Polystyrene Resin Conjugated Model Peptide with Exchange Catalyst Pd(PPh₃)₄ and with Sulfinate

Reaction 4.1 was repeated essentially as described above, except that now 0.1 eq. of Pd(PPh₃)₄ were used as the catalyst and 30-60 min. reaction time was employed for allyl/alloc deprotection. The yield of cleaved, acetylated mature peptide amounted to 82%.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above are hereby incorporated by reference.

1. Method of cyclisation of a peptide by lactamisation, comprising the steps of
a) deprotecting the peptide from allyl-type protecting groups by Pd(0) catalysis in the presence of an allyl acceptor, which peptide is protected with a base-labile protecting group at its Na and further comprises at least one allyloxy-carbonyl-protected amino-function of a lysine side chain or of an analogue of such lysine side chain and further comprises at least one allyl-ester-protected ω-carboxyl group of a glutamyl or aspartyl side chain or of an analogue of such side chain whilst retaining the base-labile protection group on the Nα, wherein said allylic protecting groups are substituted or unsubstituted,
b) cyclising the peptide by lactamisation of said deprotected side chains in the presence of a weak base reagent, and
c) deprotecting the peptide from the base-labile protection group at its Nα.

2. Method according to claim 1, characterized in that the Nα protected with a base labile protection group is the Nα of an aspartyl residue having said further allyl-ester-protected carboxyl group, and preferably wherein the aspartyl residue is part of the dipeptide sequence Asp-His.

3. Method according to claim 1, characterized in that as a further terminal step d., further elongating the peptide by means of continued peptide synthesis or peptide segment coupling of the deprotected Nα.

4. Method according to claim 3, characterized in that the peptide is bound to a solid phase during cyclisation, with the proviso that the allylic protection groups are not serving as a resin handle, and that the peptide elongation of step d. is a continued solid phase peptide synthesis.

5. Method according to claim 1, characterized in that the cylisation reaction step b. is carried out into the presence of a weak base reagent in an aprotic polar solvent and in the further presence of a coupling reagent.

6. Method according to claim 5, characterized in that the cyclisation reaction is carried out with HCTU or TCTU or with a phosphonium salt of the beuzotriazol as the coupling agent.

7. Method according to claim 6, characterized in that the peptide is linked to a resin support by means of an acid-labile bond.

8. Method according to claim 1, characterised in that the peptide is C-terminally covalently connected via an acid-labile bond.

9. Method according to claim 1, characterised in that the base-labile protection group is an FMOC group.

10. Method according to claim 1, characterised in that the allyl and alloc protection group are removed by Pd(0) catalysis in the presence of an allyl acceptor, preferably using R1—PhₙSiHₘ as the allyl acceptor that is in excess wherein R1 is a substitutent at the aromatic core and is aryl, alkyl or aralkyl, n is 1 or 2 and m is 2 or 3, more preferably wherein the allyl acceptor is PhSiH₃,

11. A cyclic peptide of formula II or III having an Nα. that is protected with a base-labile protection group, 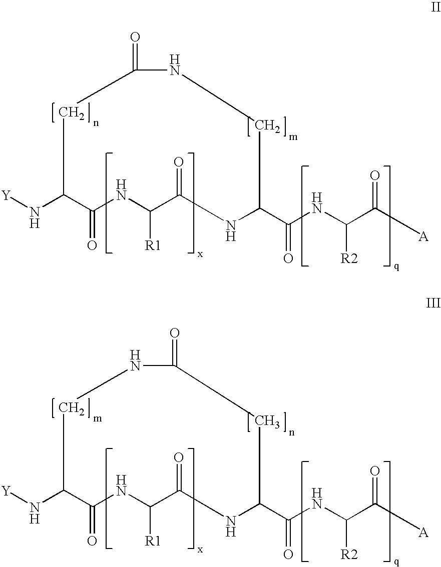
wherein Y is the base-labile protection group, n=1-10, preferably n=1 or 2, most preferably n=1, further wherein m=1-15, preferably m=3 to 6, most preferably m=3, further x=1-200 and q=0-200, preferably x=3-50 and q=0-50, wherein R1 and R2 each are a natural amino side chain or non-natural derivative thereof, which side chain further may comprise a protection group with the exception of allylether and allyloxycarbonyl protection groups, and wherein A is a resin or resin handle or wherein optionally R2 is a natural amino side chain or non-natural derivative thereof which side chain is bonded to a resin or resin handle via an ether, thioether, ester, thioester, amido or secondary or tertiary amino moiety with the proviso that then A is selected from the group consisting of OH, NH₂, NR′1H or NR′1R′2, with R′1 and R′2 being independently C₁-C₄ alkyl.

12. Peptide according to claim 11, characterized in that the base-labile group is FMOC.

13. Peptide according to claim 11, characterized in that the peptide is of the formula II with n=1 or 2, more preferably n=1.

14. Peptide according to claim 11, characterized in that A comprises a resin handle or resin linkage moiety with the exception of resin handles comprising an allyl-oxycarbonyl moiety.

15. Peptide according to claim 14, characterized in that the resin or resin handle is of formula IV 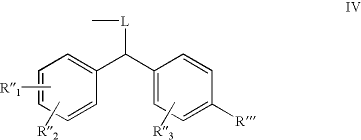
wherein R′″ is a resin, and R″1, R″2, R″3 are, independently, hydrogen, C₁-C₄ alkyl or C₁-C₄ alkoxy, and may be the same or different with the provisio that only one of R″1, R″2 may be hydrogen, and wherein L is oxygen, sulfur, nitrogen or is of formula V

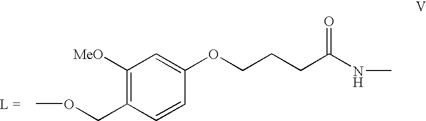

16. Peptide according to claim 15, characterized in that the resin or resin handle is of formula VI, the above definitions for radicals R′″, R″1 and R″2 applying with the proviso that L is selected from the group consisting of oxygen and nitrogen,

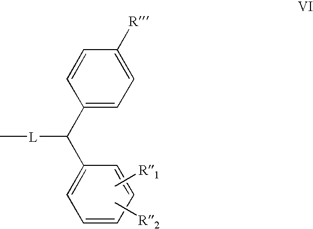

17. Peptide according to claim 16, characterized in that the resin or resin handle is of formula VII, the above definitions for radicals R″′, R″1 and R″2 applying with the proviso that L is selected from the group consisting of oxygen and nitrogen,

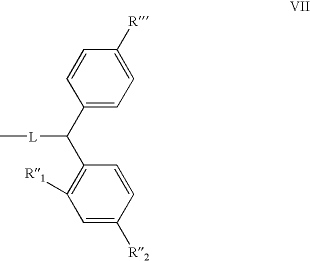

18. Peptide according to claim 17, characterized in that R″1, R″2 are, where L is oxygen, independently hydrogen, methyl or methoxy with the provisio that only one of R″1, R″2 may be hydrogen, and where L is nitrogen, independently are methyl or methoxy, preferably are methoxy.

19. Peptide according to claim 18, characterized in that L is oxygen, R″1 is hydrogen and R″2 is methyl or methoxy and preferably A is a resin or resin handle.

20. Peptide according to claim 19, characterized in that R″2 is methyl.

21. A cyclic peptide of formula II or III having an Nα that is protected with a base-labile protection group, 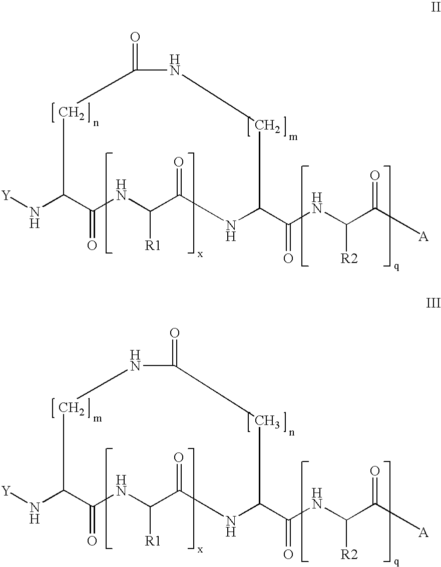
wherein Y is a base-labile protection group, n=1-10, preferably n=1 or 2, most preferably n=1, further wherein m=1-15, preferably m=3 to 6, most preferably m=3, further x=1-200 and q=0-200 wherein R1 and R2 each is, independently, a natural amino side chain or non-natural derivative thereof, which side chain further may comprise a protecting group with the exception of allylether and allyloxycarbonyl protecting groups, and wherein A is a resin or resin handle or wherein optionally R2 is a natural amino side chain or non-natural derivative thereof which side chain is bonded to a resin or resin handle via an ether, thioether, ester, thioester, amido or secondary or tertiary amino moiety with the proviso that then A is OR′3 with R′3 being a protection group with the exception of allyl groups, preferably R′3 being tert.butyl or pentafluorophenyl.

22. Use of Pd (P[ortho-tolyl]₃)₂ or Pd (P[2,4-xyloyl]₃)₂ complexes for catalyzing deprotection of allyl-protected carboxyl groups or allyloxycarbonyl protected amino and/or hydroxy groups.

23. Use according to claim 22, characterised in that an organic sulfinate is used as an allyl group acceptor or scavenger reagent.

24. Use according to claim 21, characterised in that the allyl- or allyloxycarbonyl-protected groups are part of an optionally further protected peptide, preferably an Fmoc protected peptide wherein the Fmoc group is attached to the Nα. of the peptide.

25. Method of cyclisation of a peptide by lactamisation, comprising the steps of
a. deprotecting the peptide from allyl-type protecting groups by Pd(0) catalysis in the presence of an allyl acceptor, which peptide is protected with a base-labile protecting group at its Nα. which is the Nα. of an aspartyl residue having an allyl-ester-protected ω-carboxyl group, and further comprises at least one allyloxy-carbonyl-protected amino-function of a lysine side chain or of an analogue of such lysine side chain, whilst retaining the base-labile protection group on the Nα., wherein said allylic protecting groups are substituted or unsubstituted,
b. cyclising the peptide by lactamisation of said deprotected side chains in the presence of a weak base reagent, and
c. deprotecting the peptide from the base-labile protection group at its Nα.
